(19) United States
(12) Patent Application Publication
(10) Pub. No.: US 2006/0241752 A1
(43) Pub. Date: Oct. 26, 2006

(54) Accommodating multiple lens assembly

(76) Inventor: Henry M. Israel, Bnei Brak (IL)

Correspondence Address: DEKEL PATENT LTD., DAVID KLEIN, BEIT HAROF'IM, 18 MENUHA VENAHALA STREET, ROOM 27, REHOVOT, 76209, IL

(21) Appl. No.: 11/109,703

(22) Filed: Apr. 20, 2005

(51) Int. Cl. A61F 2/16

(52) U.S. Cl. 623/6.34; 623/6.37; 623/6.43

(57) ABSTRACT

An intraocular lens assembly including a first lens and a second lens, a haptic, and a first leverage arm connecting the first lens to the haptic and a second leverage arm connecting the second lens to the haptic, the first and second lenses being arranged one in front of the other defining an anterior-posterior axis that passes perpendicularly through centers of the first and second lenses, wherein a width of the haptic extends beyond at least one of the first and second lenses in at least one direction parallel to the anterior-posterior axis.

### FIELD OF THE INVENTION

The present invention relates generally to intraocular lens (IOL) assemblies and particularly to accommodating IOL assemblies with multiple lenses.

### BACKGROUND OF THE INVENTION

Natural accommodation in a normal human eye having a normal human crystalline lens involves automatic contraction or constriction and relaxation of the ciliary muscle of the eye (and zonules controlled by the ciliary muscle) by the brain in response to looking at objects at different distances. Ciliary muscle relaxation, which is the normal state of the muscle, shapes the human crystalline lens for distant vision. Ciliary muscle contraction shapes the human crystalline lens for near vision. The brain-induced change from distant vision to near vision is referred to as accommodation.

Accommodating intraocular lens (IOL) assemblies have been developed that include an IOL that moves in response to ciliary muscular contraction and relaxation, thereby to simulate the response of the natural lens in the eye, and, inter alia, help provide patients with better focusing ability.

One example of an accommodating IOL assembly is described in U.S. Pat. No. 6,013,101 to Israel, the disclosure of which is incorporated herein by reference. The IOL assembly includes at least two, preferably rigid, linkage arms, i.e., haptics, each being attached to the optic at a first position on the arm thereof and cooperating with ciliary muscle or the zonules at a second position on the arm. There are at least two pivots, one of which is rotatably attached to each respective haptic intermediate the first and second positions.

Another example of an accommodating IOL assembly is described in U.S. Pat. No. 6,524,340 to Israel, the disclosure of which is incorporated herein by reference. In the accommodating IOL assembly of U.S. Pat. No. 6,524,340, the haptic does not apply leverage to the artificial lens. Rather one or more leverage arms, which connect the haptic to the lens, apply the requisite lever force to the lens to impart accommodating motion to the lens. In one embodiment of the invention, the leverage arm applies a lever force along a chord inwards of the perimeter of the lens. In other words, the leverage arm has a significantly greater “reach” and mechanical advantage than prior art accommodating IOLs, which rely on circumferentially attached haptics to apply leverage to the lens.

U.S. Pat. No. 6,767,363 to Bandhauer, et al. describes an accommodating intraocular lens system including a higher diopter positive intraocular lens and a lower diopter negative intraocular lens. The positive intraocular lens includes a positive optic portion having an outer peripheral edge and two or more haptic elements. The negative intraocular lens includes a negative optic portion having an outer peripheral edge and two or more haptic elements. Each haptic element is formed to have specific flexibility characteristics so as to be less resistant to bending in a plane generally parallel to an eye's optical axis than in a plane generally perpendicular to the eye's optical axis. The accommodating intraocular lens system is so designed with specific flexibility characteristics to facilitate axial displacement of the positive optic portion with respect to the negative optic portion along the eye's optical axis under a compression force. Bandhauer et al. contemplates having the positive optic portion move anteriorly with respect to the negative optic portion, with the negative optic portion either remaining in place or moving posteriorly.

### SUMMARY OF THE INVENTION

The present invention seeks to provide an improved accommodating IOL assembly in which lenses of a double or multiple lens IOL move anteriorly, as is described more in detail hereinbelow.

There is thus provided in accordance with an embodiment of the present invention an intraocular lens assembly including a first lens and a second lens, a haptic, and a first leverage arm connecting the first lens to the haptic and a second leverage arm connecting the second lens to the haptic, the first and second lenses being arranged one in front of the other defining an anterior-posterior axis that passes perpendicularly through centers of the first and second lenses, wherein a width of the haptic extends beyond at least one of the first and second lenses in at least one direction parallel to the anterior-posterior axis.

In accordance with an embodiment of the present invention the first leverage arm connects a perimeter of the first lens to the haptic and the second leverage arm connects a perimeter of the second lens to the haptic, wherein at least one of the first and second leverage arms is adapted to apply a lever force on its corresponding lens acting generally along a chord inwards of a perimeter of its corresponding lens. The first lens may be a positive lens and the second lens may be a negative lens.

There is also provided in accordance with an embodiment of the present invention an intraocular lens assembly including a first lens and a second lens, a haptic, and a first leverage arm connecting the first lens to the haptic and a second leverage arm connecting the second lens to the haptic, the first and second lenses being arranged one in front of the other defining an anterior-posterior axis that passes perpendicularly through centers of the first and second lenses, wherein the first and second leverage arms are adapted to apply leverage to and move the first and second lenses along the anterior-posterior axis to achieve a combined positive diopter accommodative effect.

### BRIEF DESCRIPTION OF THE DRAWINGS

The present invention will be understood and appreciated more fully from the following detailed description taken in conjunction with the appended drawings in which:

FIGS. 1, 2 and 3 are simplified pictorial, front view and rear view illustrations, respectively, of an intraocular lens (IOL) assembly, constructed and operative in accordance with an embodiment of the present invention;

FIG. 4A is a simplified side view illustration of the IOL assembly of FIGS. 1-3, prior to anterior displacement of first and second lenses;

FIG. 4B is a simplified side view illustration of the IOL assembly of FIGS. 1-3, in which the anterior movement of the first lens equals the anterior movement of the second lens;

FIG. 4C is a simplified side view illustration of the IOL assembly of FIGS. 1-3, in which the anterior movement of the first lens does not equal the anterior movement of the second lens;

FIG. 4D is a simplified side view illustration of the IOL assembly of FIGS. 1-3, in which a positive lens moves anteriorly and a negative lens moves posteriorly to give a total accommodative effect of a positive lens; and

FIG. 4E is a simplified side view illustration of the IOL assembly of FIGS. 1-3, in which a pair of negative lenses move posteriorly but still give a total accommodative effect of a positive lens.

### DETAILED DESCRIPTION OF THE PRESENT INVENTION

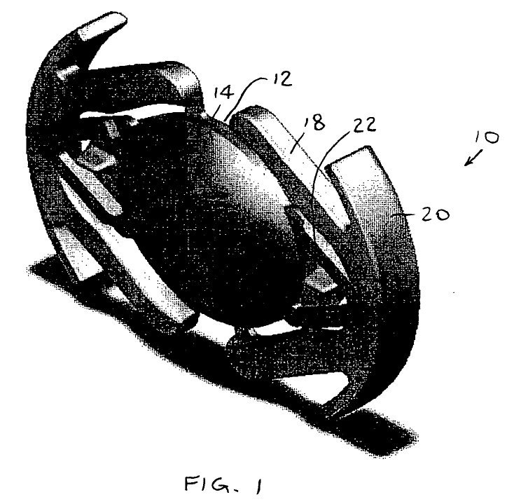

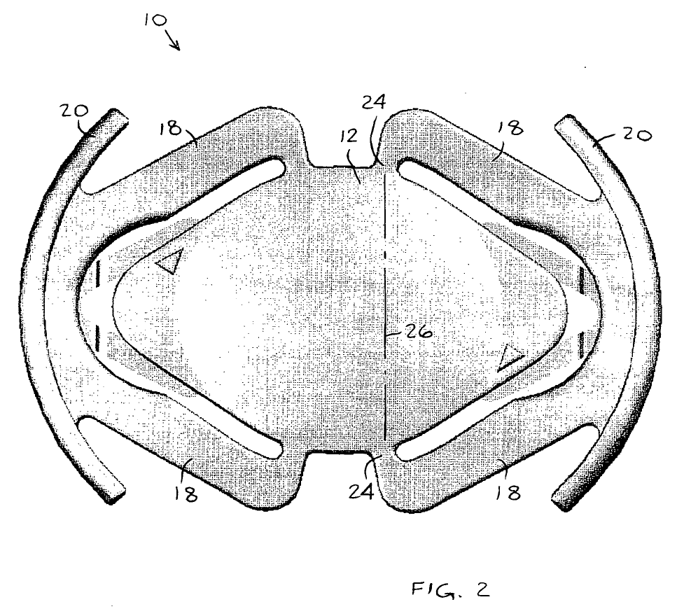

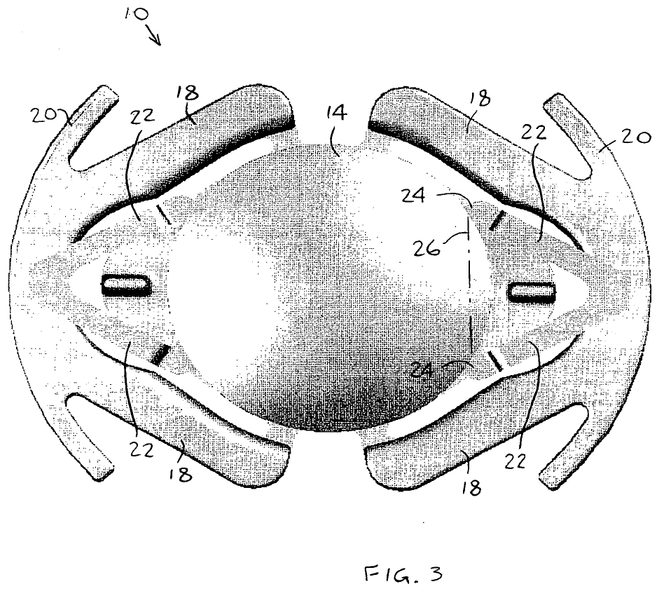

Reference is now made to FIGS. 1-4A, which illustrate an intraocular lens (IOL) assembly 10 constructed and operative in accordance with an embodiment of the present invention.

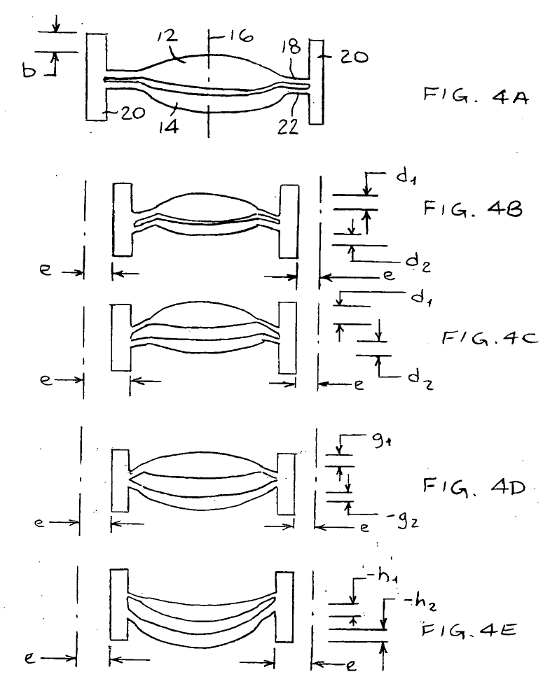

IOL assembly 10 may include a first lens 12 and a second lens 14, arranged one in front of the other defining an anterior-posterior axis 16 (FIG. 4A) that passes perpendicularly through centers of first and second lenses 12 and 14. One or more first leverage arms 18 (in the illustrated embodiment a pair of first leverage arms 18 are employed) connect first lens 12 to a haptic 20. Likewise, one or more second leverage arms 22 (in the illustrated embodiment a pair of second leverage arms 22 are employed) connect second lens 14 to haptic 20. In the illustrated embodiment, two symmetric haptics 20 are employed, shaped generally like portions of a ring. It is understood that this is just one example of a suitably shaped haptic and other sizes and shapes of haptics may be used as well. As seen in FIG. 4A, a width of haptic 20 preferably extends beyond at least one of first and second lenses 12 and 14 in at least one direction parallel to said anterior-posterior axis (this distance being indicated by letter “b”).

Haptics are clearly defined in the art as the interface elements of the IOL that touch the eye structure (“haptic” is from the Greek word for touch). The leverage arms 18 and 22 do not touch the eye structure and are not the haptics; rather they are attached to the haptics. The attachment point of the leverage arm to the haptic may be flexible, or alternatively, may be rigid with other portions of the leverage arm being flexible.

IOL assembly 10 is preferably constructed of a clear, transparent, biologically compatible material, such as but not limited to, polymethylmethacrylate (PMMA), silicone, silicone rubber, collagen, hyaluronic acid (including the sodium, potassium and other salts thereof), hydrogel, such as acrylic or methacrylic hydrogels, e.g., hydroxyethyl methacrylate or methacrylic acid copolymer/partially hydrolyzed poly(2-hydroxyethyl methacrylate) (known as PolyHEMA), polysulfones, thermolabile materials and other relatively hard or relatively soft and flexible biologically inert optical materials, or any combination of such materials, such as a gel encapsulated in a polymer. IOL assembly 10 may thus be rigid, semi-rigid or foldable, for example.

First lens 12 may be a positive lens having a higher diopter positive optic portion, e.g., approximately +20 diopter or greater, such as but not limited to +20 to +60 diopter. Second lens 14 may be a negative lens having a lower diopter negative optic portion, e.g., approximately −10 diopter or less, such as but not limited to −10 to −50 diopter. The invention also encompasses the reverse, wherein the first lens 12 is a negative lens and the second lens 14 is a positive lens, as well both being positive lenses or both being negative lenses.

As is known in the art, the number of diopters indicates the quantitative change in the distance between the lens and the focal point of light rays entering the lens. A positive diopter value describes a convex lens, while a negative value describes a concave lens. The focal point of light entering a convex lens is beyond the lens, while the focal point of light entering a concave lens is in front of the lens. A positive (convex) lens collects light rays entering the eye and reduces the distance to the focal point. A concave lens diverges light rays entering the eye, moving the true focal point of the light to a point in front of the lens.

First and second lenses 12 and 14 may be a combination of biconvex, plano-convex, plano-concave, biconcave, concave-convex (meniscus) or a diffractive-type lens of any shape, depending upon the power required to achieve the appropriate accommodative effects for multi-distance visual imaging and to achieve appropriate central and peripheral thickness for efficient handling and fit within an eye. First and second lenses 12 and 14 may be monofocal or multi-focal lenses, and they may be spherical or aspherical. They may optionally comprise a graded index of refraction, holographic (diffusing) lenses, Fresnel lenses, diffracting lenses, and/or telescopic lenses, for example.

Leverage arms 18 and 22 may be configured generally as plates or beams or other shapes. Each pair of leverage arms may be attached to its corresponding lens at two attachment zones 24, which may be generally symmetric about the center of the lens. Each leverage arm may apply a lever force on its corresponding lens acting generally along a chord 26 inward of the perimeter of the lens 12 or 14. The chord 26 preferably connects the two attachment zones 24. Specifically, each leverage arm 18 or 22 acts as a torque or moment transfer device that transfers ciliary muscle relaxation or contraction (designated as distance e in FIGS. 4B and 4C) into a force on the corresponding lens, which causes that lens to generally translate either anteriorly for near vision (generally as a result of ciliary muscle contraction) or posteriorly for distant vision (ciliary muscle relaxation), generally along the anterior-posterior axis 16. Each leverage arm 18 or 22 has a significantly greater “reach” and mechanical advantage than prior art accommodating IOLs, which rely on circumferentially attached haptics to apply leverage to the lens. The force transferred by the leverage arms 18 and 22 is generally independent of the boundary condition of haptics 20.

Unlike the prior art, both first and second lenses 12 and 14 are adapted to move anteriorly along the anterior-posterior axis 16 to achieve an accommodative effect. This is in contrast to the prior art of U.S. Pat. No. 6,767,363 to Bandhauer, et al., wherein the positive optic portion moves anteriorly with respect to the negative optic portion, with the negative optic portion either remaining in place or moving posteriorly. Nevertheless, as will be described below, the present invention does contemplate having a positive lens move anteriorly and a negative lens move posteriorly or two negative lenses moving posteriorly, wherein in all cases the total effect is one of a positive lens, that is, reducing the distance to the focal point. In Bandhauer, et al., the haptics move the lenses without leverage. In contrast, in the present invention, leverage arms 18 and 22 apply leverage to and move the lenses 12 and 14, respectively. The combined sum of the lenses is positive in all locations within the accommodative motion.

Natural brain-induced accommodating forces within the eye cause a contractive displacement “e” on the haptics 20 and leverage arms 18 and 22. This causes the leverage arms 18 and 22 to flex or otherwise move in the anterior direction, thereby moving the lenses anteriorly. FIGS. 4B and 4C illustrate the anterior motion of the lenses. The anterior movement of the first lens 12 is designated as d₁, whereas the anterior movement of the second lens 14 is designated as d₂. In FIG. 4B, d₁=d₂, meaning that the distance between the first and second lenses 12 and 14 does not change as the lenses move anteriorly. In contrast, making reference to FIG. 4C, d₁≠d₂, meaning that the distance between the first and second lenses 12 and 14 changes as the lenses move anteriorly. The difference influences the amount of diopters obtained in the accommodative effect.

Reference is now made to FIG. 4D. In this embodiment, the front (positive) lens 12 moves anteriorly and the second (negative) lens 14 moves posteriorly. The anterior movement of the first lens 12 is designated as g₁, whereas the posterior movement of the second lens 14 is designated as −g₂. Their motions contribute to a total accommodative effect of a positive lens, i.e., reduction of the distance to the focal point.

Reference is now made to FIG. 4E. In this embodiment, the front (negative) lens 12 moves posteriorly and the second (negative) lens 14 also moves posteriorly. The posterior movement of the first lens 12 is designated as −h₁, whereas the posterior movement of the second lens 14 is designated as −g₂. Their motions contribute to a total accommodative effect of a positive lens, i.e., reduction of the distance to the focal point.

As seen by comparing the first and second leverage arms 18 and 22, the leverage arms 18 and 22 may have different lengths. They may be attached at different points along peripheries of the first and second lenses 12 and 14, respectively. The different lengths and attachment points may be chosen to produce different effects, such as different anterior displacements, rotations of the lenses (e.g., about axis 16 or other axes) or lateral displacements of the lenses.

It will be appreciated by persons skilled in the art that the present invention is not limited by what has been particularly shown and described hereinabove. Rather the scope of the present invention includes both combinations and subcombinations of the features described hereinabove as well as modifications and variations thereof which would occur to a person of skill in the art upon reading the foregoing description and which are not in the prior art.

What is claimed is:

1. An intraocular lens assembly comprising:
a first lens and a second lens;
a haptic; and
a first leverage arm connecting said first lens to said haptic and a second leverage arm connecting said second lens to said haptic, said first and second lenses being arranged one in front of the other defining an anterior-posterior axis that passes perpendicularly through centers of said first and second lenses, wherein a width of said haptic extends beyond at least one of said first and second lenses in at least one direction parallel to said anterior-posterior axis.

2. The intraocular lens assembly according to claim 1, wherein both said first and second lenses are adapted to move anteriorly along said anterior-posterior axis to achieve an accommodative effect.

3. The intraocular lens assembly according to claim 1, wherein said first leverage arm connects a perimeter of said first lens to said haptic and said second leverage arm connects a perimeter of said second lens to said haptic, wherein at least one of said first and second leverage arms is adapted to apply a lever force on its corresponding lens acting generally along a chord inwards of a perimeter of its corresponding lens.

4. The intraocular lens assembly according to claim 1, wherein said first lens is a positive lens and said second lens is a negative lens.

5. The intraocular lens assembly according to claim 4, wherein said positive lens comprises a higher diopter positive optic portion, and said negative lens comprises a lower diopter negative optic portion.

6. The intraocular lens assembly according to claim 2, wherein a distance between said first and second lenses does not change as said lenses move anteriorly.

7. The intraocular lens assembly according to claim 2, wherein a distance between said first and second lenses changes as said lenses move anteriorly.

8. The intraocular lens assembly according to claim 1, wherein said first and second leverage arms have different lengths.

9. The intraocular lens assembly according to claim 1, wherein said first and second leverage arms are attached at different points along peripheries of said first and second lenses, respectively.

10. An intraocular lens assembly comprising:
a first lens and a second lens;
a haptic; and
a first leverage arm connecting said first lens to said haptic and a second leverage arm connecting said second lens to said haptic, said first and second lenses being arranged one in front of the other defining an anterior-posterior axis that passes perpendicularly through centers of said first and second lenses, wherein both said first and second lenses are adapted to move anteriorly along said anterior-posterior axis to achieve an accommodative effect.

11. The intraocular lens assembly according to claim 10, wherein said first leverage arm connects a perimeter of said first lens to said haptic and said second leverage arm connects a perimeter of said second lens to said haptic, wherein at least one of said first and second leverage arms is adapted to apply a lever force on its corresponding lens acting generally along a chord inwards of a perimeter of its corresponding lens.

12. The intraocular lens assembly according to claim 10, wherein said first lens is a positive lens and said second lens is a negative lens.

13. The intraocular lens assembly according to claim 10, wherein said first lens is a negative lens and said second lens is a positive lens.

14. The intraocular lens assembly according to claim 10, wherein a distance between said first and second lenses does not change as said lenses move anteriorly.

15. The intraocular lens assembly according to claim 10, wherein a distance between said first and second lenses changes as said lenses move anteriorly.

16. The intraocular lens assembly according to claim 10, wherein said first and second leverage arms have different lengths.

17. The intraocular lens assembly according to claim 10, wherein said first and second leverage arms are attached at different points along peripheries of said first and second lenses, respectively.

18. An intraocular lens assembly comprising:
a first lens and a second lens;
a haptic; and
a first leverage arm connecting said first lens to said haptic and a second leverage arm connecting said second lens to said haptic, said first and second lenses being arranged one in front of the other defining an anterior-posterior axis that passes perpendicularly through centers of said first and second lenses, wherein said first and second leverage arms are adapted to apply leverage to and move said first and second lenses along said anterior-posterior axis to achieve a combined positive diopter accommodative effect.
